(19) United States
(12) Patent Application Publication
(10) Pub. No.: US 2020/0113249 A1
(43) Pub. Date: Apr. 16, 2020

(54) POSTPARTUM BODY SUPPORT BINDER

(71) Applicants: Aznizah Binti Zainal Abidin, Cheras (MY); Fareen Shazli Bin Ali, Cheras (MY)

(72) Inventors: Aznizah Binti Zainal Abidin, Cheras (MY); Fareen Shazli Bin Ali, Cheras (MY)

(21) Appl. No.: 16/561,064

(22) Filed: Sep. 5, 2019

(30) Foreign Application Priority Data
Sep. 5, 2018 (MY) PI2018703146

(51) Int. Cl. A41C 1/10; A61F 13/14

(52) U.S. Cl. CPC A41C 1/10; A61F 13/148

(57) ABSTRACT

The present invention provides a body support binder (100) for correcting diastasis recti and pubic symphasis separation adapted to encircle and support abdomen and thigh regions. The body support binder comprises a front section (104) and a rear section (102) and a removable fastener affixed to a fastener panel (106) on the front side characterized in that the rear section (102) having at least two stretching directions preferably crosswise and lengthwise and the front section (104) stretches in one direction preferably from selvedge to selvedge.

### FIELD OF INVENTION

The present invention relates to body supports and binders and more particularly postpartum body support binder.

### BACKGROUND ART

Post-partum binders are well known in the art for use by new mothers to regain their body shape after childbirth. Traditionally, within the Malay community of Southeast Asia region, post-partum binders also known as ‘bengkung’ are helps to provide spinal support for good posture, flattening of the stomach, reducing abdominal muscles separation (diastasis recti) shrinking of the uterus and tightening of birth canal during postnatal period. Essentially, a ‘bengkung’ is a long piece of cloth cinctured snugly around abdominal and hip regions worn together with medicinal herbs, spices and medicated oils to accelerate the process of regaining post pregnancy body shape. There are various types of traditional ‘bengkung’ which includes ‘bengkung barut’ also known as bandage bengkung which is provided strings on the left and right sides to be tied together; ‘bengkung zigzag’ is corset style binder tied with strings in a criss-cross manner and ‘bengkung Java’ which is a long cloth wrapped around the torso.

However, these traditional binders (i.e ‘bengkung’) posses a myriad of challenges to wearers such as requiring felicitous binding skill; uncomfortable material; tedious binding process and inadequate material length to sufficiently wrap around the wearer's body and thereby fails to provide appropriate support and compression on the desired body areas.

A number of patented technologies are disclosed as body binders or belly braces for post partum use with the aim for providing means for re-gaining pre-pregnancy body state. An example of such a belly brace is disclosed in Chinese Utility Model No. CN204542497 (U) (hereinafter referred to as the CN 497 Utility Model) entitled “Restraint Abdomen Corset Postpartum” having a filing date of 9 Feb. 2018 (Applicant: SHANGHAI SUNWIN INDUSTRY CO., LTD and SHANGHAI I-BABY CO., LTD). The CN 497 Utility Model provides a restraint abdomen postpartum corset for the recovery of postpartum associated bodily changes such as visceral ptosis and pelvic widening, The CN 497 Utility Model comprises of belt body and body knitted with shaping belt, corset belt, closed belt, belt and crotch belt. The CN 497 Utility Model applies synergistic pressure at specific parts of the body which accelerates the progress of post-partum body recovery.

Another example of belly brace is disclosed in U.S. Pat. No. 2,405,711 B2 (hereinafter referred to as US 711 Patent) entitled “Corset” having a filing date of 20 Jan. 1945 (Applicant: Even-Pul Foundations, Inc.). The US 711 Patent discloses improved garment adapted for effectively reducing the abdomen and keeping the waist and hip in proper form without discomfort to the wearer. The US 711 Patent comprises of two similar sections united by lacing at the back and adapted to be connected by separable fasteners at the front, each section including a back portion and a front portion, and a belt located on the inner face of the front portion of each section, each belt including a central elastic portion and side inelastic portions attached to the front portion of the associated section. The US 711 Patent is fitted snugly to the wearer as to have the desired reducing and form-keeping qualities without causing discomfort to the wearer.

Yet another example is disclosed in U.S. Pat. No. 3,970,079 X6 (hereinafter known as US 079 Patent) entitled “Body Support Binder and Method of Fabricating Same” having a filing date of 21 Mar. 1975 (Applicant: Medical Specialities, Inc.). The US 079 Patent discloses an elongate body support binder adapted to encircle and reinforce a portion of the body of a wearer. The US 079 Patent is characterised by the ability to resist rolling of the longitudinal side edges when in use without employing separate stiffeners and the like and which also eliminates the need for forming tucks or pockets to accommodate such stiffeners or stays. The US 079 Patent is in the form of thoracic belt configured to place about the thorax of the wearer so as to conform more readily to the shape of that portion of the wearer's body.

Although the above-mentioned prior arts provide various functional enhancements in body binders there is a continued need to improvise the fabric, length, structure and shape of the existing technologies to provide effective, supportive and comfortable body wraps.

### SUMMARY OF INVENTION

The present invention relates to body supports and binders, more particularly to body support binders for postpartum use.

The following presents a simplified summary of the invention in order to provide a basic understanding of some aspects of the invention. This summary is not an extensive overview of the invention. Its sole purpose is to present some concepts of the invention in a simplified form as prelude to the more detailed description that is presented later.

One aspect of the invention provides a body support binder (100) for correcting diastasis recti and pubic symphasis separation adapted to encircle and support abdomen and thigh regions. The body support binder comprising a front section (104) to provide compression and a rear section (104) to provide support; and a removable fastener affixed to a fastener panel (106) on the front section. The rear section (102) having at least two stretching directions and the front section (104) stretching in at least one direction.

Another aspect of the invention provides that the front section (104) is configured to provide contraction in the hip, belly, thighs and buttocks region of a user.

A further aspect of the invention provides the stretching direction of the front section (104) is selvedge to selvedge and the rear section (102) stretches crosswise and lengthwise.

Still another aspect of the invention provides that the binder is an elongate binder which provides support to vertical axis of the abdomen and returns rectus abdominal muscles to its original body position or structure.

Yet another aspect of the invention provides that the fastener is preferably a string or a plurality of springs, zip or buttons to annex the binder from a bottom thigh region to a lower chest region.

A further aspect of the invention provides that the length of the binder enables wraparound from a bottom thigh region to a lower chest region of a user.

Yet another aspect of the invention provides that the binder is for post-partum use.

Still another aspect of the invention provides that the binder is for use in regaining body shape.

A further aspect of the invention provides that the material for the binder is preferably made of stretchable, breathable nano functional textiles with water repellent and anti-microbial properties.

Another aspect of the invention provides that the binder is substantially elongated with two curvature portions at each ends of the binder.

The present invention consists of features and a combination of parts hereinafter fully described and illustrated in the accompanying drawings, it is being understood that various changes in the details may be made without departing the scope of the invention or sacrificing any of the advantages of the present invention.

### BRIEF DESCRIPTION OF ACCOMPANYING DRAWINGS

To further clarify various aspects of some embodiments of the present invention, a more particular description of the invention will be rendered by references to specific embodiments thereof, which are illustrated in the appended drawings. It is appreciated that these drawings depict only typical embodiments of the invention and are therefore not to be considered limiting of its scope. The invention will be described and explained with additional specificity and detail through the accompanying drawings.

FIG. 1.0 illustrates a two dimensional line drawing of the body support binder of the present invention.

FIGS. 2.0 (a), (b), (c) and (d) illustrate multiple views of an embodiment of the present invention.

### DETAILED DESCRIPTION OF THE PREFERRED EMBODIMENTS

The present invention relates to body supports and binders, and more particularly postpartum body support binder.

In Asia, body binders known as bengkung are traditionally used by new mothers to regain the pre-pregnancy body structure. These body binders provide apt spinal support and stability to a user's abdomen and pelvic regions as well as aids in tightening the birth canal. However, the conventional body support binders are often bulky, uncomfortable, and difficult to adjust for a proper supportive fit.

The present invention provides a body support binder which overcomes the above-mentioned shortcomings by providing an elongate binder with at least two stretching directions to prevent fat from converging around the abdomen and hip regions and further tightening the said region to allow the user to regain original body shape.

Hereinafter, this specification will describe the present invention according to the preferred embodiments. It is to be understood that limiting the description to the preferred embodiments of the invention is merely to facilitate discussion of the present invention and it is envisioned without departing from the scope of the appended claims.

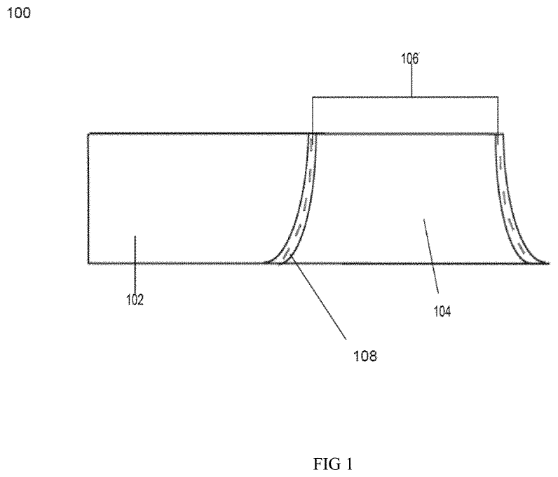

FIG. 1.0 illustrates a body support binder (100) of the present invention which comprises a front section (104), a rear section (102) and a removable fastener (108) affixed to a fastener panel (106) on the front section of the binder. The front section (104) is configured to provide compression to a user while the rear section (104) provides support. The body support binder is an elongated binder adapted to encircle a user's lower chest to thigh region for correcting diastasis recti and pubic symphasis separation. Further, the body support binder provides support to a vertical axis of the abdomen to effectively return rectus abdominal muscles to its original body structure. The fastener (108) is preferably a string or a plurality of strings, hooks, straps, zip or buttons to annex the binder from bottom thigh region to lower chest region. The fastener is affixed to the fastener panel positioned in vertically over user's abdominal midline. The fastener is highly adjustable to provide varying levels of support to the abdominal, pelvic and gluteus regions. The binder is preferably continuously elongated and in rectangular shape with curvature portions on each ends of the binder. The length of the binder is adequately long to allow wraparound from the lower region of the chest to the region of the thighs of a user.

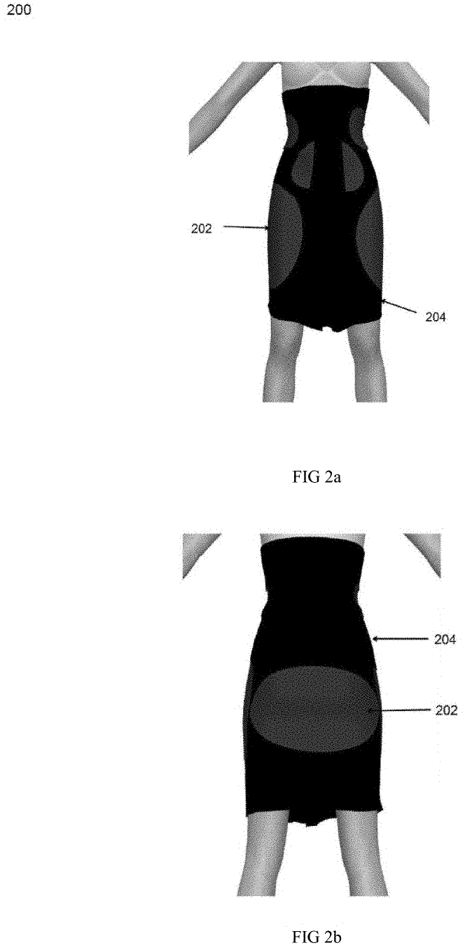

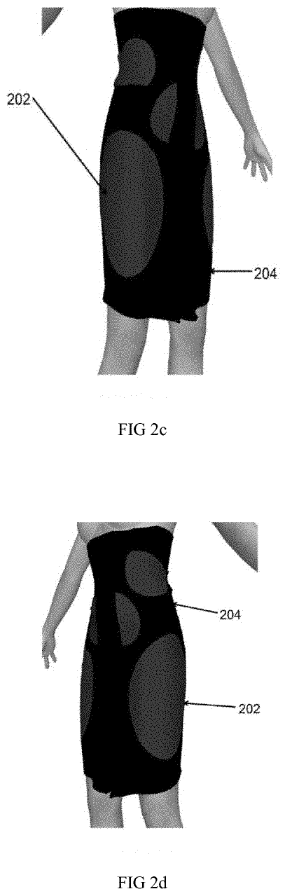

FIGS. 2 (a) to 2 (d) illustrate the multiple views of the different compression areas (202) and support areas (204) provided by the binder. The front section (104) provides contractions to the compression areas (202) such as hip, belly, thigh and gluteus regions of a user. The said contractions prevent fat from converging around the hip, belly, thigh and gluteal regions. The front section (104) is configured to provide stretching in one direction. Preferably, the stretching direction is from selvedge to selvedge. This feature restricts the movements in specific compression areas (202) of belly, thighs, sides of abdomen and buttocks. The rear section (102) is configured to provide at least two directional stretching. Preferably, the stretching directions of the rear section (102) are crosswise and lengthwise which stretches in four (4) directions. This feature provides support and allows movements and relieves discomfort in the abdominal region of a user. FIG. 2 (a) illustrates the front view of compression areas (202) provided by the binder, preferably on the sides of thighs, abdomen and belly. FIG. 2 (b) illustrates the rear view of the body support binder. The compression area (202) is predominantly concentrated on the gluteus region to provide contraction and tightening. FIGS. 2 (c) and (d) illustrate the side views of the body support binder. The compression areas (202) allow body shaping in belly, thigh, and gluteus regions while the support areas (204) provide free movements and reduces discomfort to the user.

The body support binder is suitable for providing rehabilitation to mothers after a caesarean section procedure or natural birthing. Further, the binder may also be used by anyone wanting to regain original body shape due to diastasis recti and pubic symphasis separation. The material is light-weighted, breathable and constructed to provide anti-microbial, water repellent and odour control properties. Suitable materials of the binder include, but not limited to, nano functional textiles, nylon, spandex, darlex, Lycra™, jersey, cotton, polyurethane, neoprene, elastomer and the like. The material may be a breathable fabric. Preferably, the material is constructed from nano functional textiles providing at least two-way stretch fabrics. The stretching directions may be selvedge to selvedge, crosswise and lengthwise. The body binder is fabricated in different sizes to cater for users with different body shapes and sizes. The sizing of the body support binder is dependent on the type of materials as well as the height and weight of the user. As illustrated in FIG. 1.0, the x axis of the material denotes the length of the material while the y axis of the material denotes the width of the material. The y axis which denotes the width enables to “cover” from the bottom thigh region of the user to a lower chest region of a user while the x axis which denotes the length enables “wraparound” from a bottom thigh region to a lower chest region of a user.

Further, the body binder is versatile for use as support to the organs and muscles, to reduce motion and stabilize joints of the lower back or pelvis.

Unless the context requires otherwise or specifically stated to the contrary, integers, steps or elements of the invention recited herein as singular integers, steps or elements clearly encompass both singular and plural forms of the recited integers, steps or elements.

Throughout this specification, unless the context requires otherwise, the word “comprise”, or variations such as “comprises” or “comprising”, will be understood to imply the inclusion of a stated step or element or integer or group of steps or elements or integers, but not the exclusion of any other step or element or integer or group of steps, elements or integers. Thus, in the context of this specification, the term “comprising” is used in an inclusive sense and thus should be understood as meaning “including principally, but not necessarily solely”.

What is claimed is:

1. A body support binder (100) for correcting diastasis recti and pubic symphasis separation adapted to encircle and support both abdomen and thigh regions comprises:
a front section (104) to provide compression;
a rear section (102) to provide support; and
a removable fastener affixed to a fastener panel (106) on the front section;
characterized in that
the rear section (102) further having at least two stretching directions and the front section (104) having at least one stretching direction.

2. The body support binder (100) according to claim 1, wherein the front section (104) is configured to provide contraction in the hip, belly, thighs and buttocks region of a user.

3. The body support binder (100) according to claim 1, wherein the stretching direction of the front section (104) is selvedge to selvedge and the rear section (102) stretches crosswise and lengthwise.

4. The body support binder (100) according to claim 1, wherein the binder is an elongate binder which provides support to a vertical axis of the abdomen and returns rectus abdominal muscles to its original body position or structure.

5. The body support binder (100) according to claim 1, wherein the fastener is preferably a string or a plurality of strings, hooks, straps, zip or buttons to annex the binder from a bottom thigh region to a lower chest region.

6. The body support binder (100) according to claim 1, wherein the length of the binder enables wraparound a bottom thigh region to a lower chest region of a user.

7. The body support binder (100) according the claim 1, wherein the binder is for post-partum use.

8. The body support binder (100) according to claim 1, wherein the binder is for use in regaining body shape.

9. The body support binder (100) according to claim 1, wherein the material for the binder is preferably made of stretchable, breathable nano functional textiles with water repellent and anti-microbial properties.

10. The body support binder (100) according to claim 1, wherein the binder is substantially elongated with two curvature portions at each ends of the binder.
